(19) United States
(12) Patent Application Publication
(10) Pub. No.: US 2024/0207070 A1
(43) Pub. Date: Jun. 27, 2024

(54) BIONIC PROSTHESIS PERCEIVING THE ENVIRONMENT

(71) Applicant: Luca Miller, Paris (FR)

(72) Inventor: Luca Miller, Paris (FR)

(21) Appl. No.: 18/555,941

(22) PCT Filed: Apr. 14, 2022
(86) PCT No.: PCT/EP2022/060115

§ 371 Date: Oct. 18, 2023

(30) Foreign Application Priority Data
Apr. 19, 2021 (FR) FR2104027

(51) Int. Cl. A61F 2/58; A61F 2/66; A61F 2/68; A61F 2/72

(52) U.S. Cl. CPC A61F 2/586; A61F 2/66; A61F 2/72; A61F 2002/6614; A61F 2002/6881

(57) ABSTRACT

The present invention relates to a bionic prosthesis of the lower or upper limb, having a prosthetic distal segment, in other words a prosthetic foot or a prosthetic hand, adapted to come into contact with a terrain or with an object to be grasped; a prosthetic middle segment, in other words a prosthetic tibia or a prosthetic forearm; at least one motorized articulation device, of which one is connected mechanically to the prosthetic distal segment and to the prosthetic middle segment; at least one depth camera adapted to generate a three-dimensional point cloud; a control unit configured to process the three-dimensional point cloud and to control said at least one motorized articulation device, so as to adapt the positioning of the prosthetic distal segment with respect to the terrain or to the object to be grasped.

### TECHNICAL FIELD

The present invention relates to the field of bionic prostheses intended for limb amputees. In particular, the present invention provides a bionic lower or upper limb prosthesis perceiving the environment and a method for controlling said bionic prosthesis.

### BACKGROUND

As is known, a prosthesis allows a limb to be replaced from a functional and esthetic point of view. Within the context of a lower limb amputee, it is desirable to have a prosthesis that makes walking as natural as possible and allows the user to maintain their balance, especially for example when climbing and descending stairs, or when walking backwards. Similarly, within the context of an upper limb amputee, it is desirable to facilitate the gripping of objects, taking account especially of the shape and nature of the object to be gripped. By way of example, the optimal clamping force and position of the prosthesis differ for gripping a glass and gripping a fruit.

In particular, a bionic prosthesis distinguishes itself in that it implements a robotic process that, based on commands corresponding to the intentions of the amputee, triggers a motion of the prosthesis. Different categories of bionic prostheses are documented in the state of the art, the most common being myoelectric prostheses, hydraulic prostheses, neuroelectric prostheses, and electronic prostheses.

As is known, myoelectric prostheses rely on muscle activation. Sensors, especially at the stump, detect muscle contractions and send electrical signals in order to control the prosthesis. The amputee's handling of this prosthesis requires extensive reeducation work in order to learn how to adequately control muscle contractions. Thus, the use of a myoelectric prosthesis requires perfect control of the amputee to be able to trigger the motions with adequate precision and force. In addition, the possible motions are generally limited.

According to another known example, electronic prostheses have pre-programmed actions that the amputee can activate especially via a mobile application.

Consequently, according to the two previous examples, motion is the result of the amputee's will and considered actions. In addition to the difficulty of achieving precise control of the motions performed, another drawback lies in the mental load that the control of this type of prosthesis implies for the user.

There is therefore a need for bionic prostheses that allow more natural movement, especially without requiring heavy learning or significant mental load for the user.

In addition, bionic prostheses may comprise resistance measurement means in order to adapt the motion control. Thus, bionic prostheses according to the state of the art are mainly reactive, in other words they have limited awareness of the external environment. All possible motions are limited accordingly.

Therefore, there is a need for a bionic prosthesis with improved autonomy and precision, especially a bionic prosthesis that perceives the environment autonomously.

### SUMMARY

More precisely, according to one aspect of the invention, one object of the invention is a bionic lower or upper limb prosthesis, comprising:
a prosthetic distal segment of the lower or upper limb, in other words, a prosthetic foot or, respectively, a prosthetic hand, at a free end of the bionic prosthesis, adapted to contact a ground or, respectively, an object to be gripped;
a prosthetic middle segment, in other words, a prosthetic tibia or, respectively, a prosthetic forearm;
at least one motor-driven hinge device, a first of said at least one motor-driven hinge device being mechanically connected to the prosthetic distal segment on the one hand and to the prosthetic middle segment on the other hand;
a control unit configured to control said at least one motor-driven hinge device from control signals corresponding to a motion intention;
at least one depth camera adapted to generate a three-dimensional point cloud of the environment of the bionic prosthesis, comprising the ground or, respectively, the object to be gripped,

Then, the control unit is configured to process the three-dimensional point cloud in order to detect the ground or, respectively, the object to be gripped, and to control said at least one motor-driven hinge device so as to adapt the positioning of the prosthetic distal segment relative to the ground or, respectively, relative to the object to be gripped. The use of depth cameras offers the considerable advantage of improving the perception of the external environment and therefore improving the movement of the bionic prosthesis by supplementing the unconscious sensory perceptions of the amputee. The present invention further enables the implementation of motor-driven hinge devices having a higher number of degrees of freedom, making the movement of the bionic prosthesis more natural.

Advantageously, the bionic prosthesis according to the invention comprises a myoelectric sensor system configured to generate the control signals corresponding to a motion intention.

According to a first embodiment of the invention, the bionic prosthesis forms a bionic lower limb prosthesis, comprising a prosthetic foot at a free end of the bionic prosthesis, adapted to contact a ground, a prosthetic tibia, at least one motor-driven hinge device, a control unit configured to control said at least one motor-driven hinge device from control signals corresponding to a motion intention, and at least one depth camera adapted to generate a three-dimensional point cloud of the environment of the prosthetic foot comprising the ground. A first of said at least one motor-driven hinge device is mechanically connected to the prosthetic foot on the one hand and to the prosthetic tibia on the other hand. In addition, the control unit is configured to process the three-dimensional point cloud in order to detect the ground and to control said at least one motor-driven hinge device so as to adapt the positioning of the prosthetic foot relative to the ground.

Advantageously, said at least one depth camera consists of two depth cameras configured to cover respectively a front field and a rear field of the environment of the prosthetic foot.

Advantageously, the prosthetic foot having a directional vector of the foot oriented substantially in a longitudinal direction of the prosthetic foot, the bionic lower limb prosthesis comprises a set of accelerometers capable of measuring the directional vector of the foot.

According to one example of the invention, the bionic lower limb prosthesis according to the first embodiment of the invention comprises a prosthetic femur, said at least one motor-driven hinge device then comprising a second motor-driven hinge device being mechanically connected to the prosthetic tibia on the one hand and to the prosthetic femur on the other hand.

According to another aspect of the invention, the invention relates to a method for controlling the bionic lower upper or limb prosthesis according to the invention, the method, following an instruction corresponding to a motion intention and comprising:
measuring the current state of the bionic prosthesis comprising especially measuring directional vectors of the prosthetic distal segment, the prosthetic middle segment, and, where applicable, the prosthetic proximal segment;
acquiring a three-dimensional point cloud of the environment of the bionic prosthesis by said at least one depth camera;
processing the three-dimensional point cloud by the control unit;
calculating the motions to be implemented by said at least one motor-driven hinge device by the control unit;
moving the bionic prosthesis.

Advantageously, the method for controlling a bionic lower limb prosthesis according to the first embodiment of the invention, following an instruction corresponding to a motion intention, comprises:
measuring the current state of the bionic prosthesis comprising measuring a directional vector of the foot oriented substantially in a longitudinal direction of the prosthetic foot;
acquiring the three-dimensional point cloud of the environment of the bionic prosthesis by said at least one depth camera;
processing the three-dimensional point cloud by the control unit comprising extracting a set of points from the three-dimensional point cloud corresponding to the ground and calculating the normal to a surface defined by said set of points, referred to as the normal to the ground;
calculating the motions to be implemented by said at least one motor-driven hinge device by the control unit so that the directional vector of the foot is substantially perpendicular to the normal to the ground;
moving the bionic prosthesis.

Advantageously, the method according to the invention comprises a step of reconstructing the three-dimensional point cloud comprising assembling a plurality of three-dimensional point clouds corresponding to successively generating three-dimensional point clouds by said at least one depth camera, the reconstruction step being performed prior to calculating the normal to the ground.

Advantageously, measuring the directional vector of the foot is performed by a set of accelerometers.

### BRIEF DESCRIPTION OF THE DRAWINGS

The invention will be better understood upon reading the following description, given solely by way of example, and by referring to the accompanying figures, given by way of non-limiting examples, in which identical references are given to similar objects and in which:

FIG. 1 is a schematic representation of a view of the bionic lower limb prosthesis according to one aspect of a first embodiment of the invention;

FIG. 2 is a schematic representation of the method for controlling the bionic lower limb prosthesis according to another aspect of the first embodiment of the invention;

FIG. 3 is a schematic representation of the method for controlling the bionic upper limb prosthesis according to one aspect of a second embodiment of the invention.

It should be noted that the figures set out the invention in detail for implementing the invention, said figures can of course be used to better define the invention where applicable.

### DETAILED DESCRIPTION

According to one aspect of the invention, the invention relates to a bionic prosthesis and will be described below within the context of a myoelectric bionic prosthesis for a lower or upper limb amputee. Since the invention is not limited to this scope, it may also be adapted to any bionic prosthesis of the lower or upper limb.

The bionic prosthesis according to the invention comprises a prosthetic distal segment of the lower or upper limb, in other words, a prosthetic foot or, respectively, a prosthetic hand, at a free end of the bionic prosthesis, and adapted to contact a ground or, respectively, an object to be gripped; a prosthetic middle segment, in other words, a prosthetic tibia or, respectively, a prosthetic forearm; at least one motor-driven hinge device, a control unit, and at least one depth camera.

A first of said at least one motor-driven hinge device is mechanically connected to the prosthetic distal segment on the one hand and to the prosthetic middle segment on the other hand. The first motor-driven hinge device corresponds in particular to the ankle joint for a bionic lower limb prosthesis and the wrist joint for a bionic upper limb prosthesis.

The control unit is configured to control said at least one motor-driven hinge device from control signals corresponding to a motion intention. Preferably, the control unit implements the ROS (“Robotic Operation System”) or ROS 2 operating system.

In order to connect the bionic prosthesis according to the invention to the amputee, the bionic prosthesis may advantageously comprise an interlocking member adapted to be added to a residual end of an amputated limb. Alternatively, it is also possible to connect the bionic prosthesis to the amputee by an implant system, in particular an OPRA implant system, which is placed by an osteointegration process.

Within the context of a myoelectric bionic prosthesis, the bionic prosthesis especially comprises a myoelectric sensor system configured to generate the control signals corresponding to a motion intention. The control signals may in particular be a simple command, such as a command to walk for a lower limb prosthesis, or to grip an object for an upper limb prosthesis. The myoelectric sensor system may also enable more advanced control of the prosthesis, especially by being capable of capturing a speed and run time associated with the motion intention, so as to allow more precise motion, in the order of a few degrees, and at a low speed. Within this context, the motion intention corresponds to muscle contractions of the amputee that are detected by the myoelectric sensor system. The myoelectric sensor system can be integrated into the interlocking member where applicable.

Said at least one depth camera is adapted to generate a three-dimensional point cloud of the environment of the bionic prosthesis, comprising the ground, or, respectively, the object to be gripped. Then, the control unit is configured to process the three-dimensional point cloud in order to detect the ground or, respectively, the object to be gripped, and to control said at least one motor-driven hinge device so as to adapt the positioning of the prosthetic distal segment relative to the ground or, respectively, relative to the object to be gripped.

Thus, the use of depth cameras advantageously makes it possible to improve the perception of the external environment and therefore to improve the movement of the bionic prosthesis by supplementing the unconscious sensory perceptions of the amputee. Consequently, the present invention has the considerable advantage of enabling the implementation of motor-driven hinge devices having a higher number of degrees of freedom, making the movement of the bionic prosthesis more natural.

In particular, a depth camera, also referred to as a Time of Flight (TOF) camera, comprises two infrared depth sensors for detecting distances and shapes in three-dimensional space.

In addition, said at least one depth camera is, preferably, activated only when the bionic prosthesis is moved and otherwise is at rest. This operation advantageously makes it possible to reduce the power consumption of the bionic prosthesis.

Furthermore, the bionic prosthesis may, where applicable, comprise a prosthetic proximal segment. The proximal prosthesis segment corresponds in particular to a prosthetic femur for a lower limb amputation, or respectively, a prosthetic arm for an upper limb amputation. In addition, said at least one motor-driven hinge device then preferably comprises a second motor-driven hinge device corresponding to the knee joint for a lower limb amputation, and the elbow joint for an upper limb amputation.

The bionic prosthesis preferably comprises a set of accelerometers capable of measuring a current state of the bionic prosthesis.

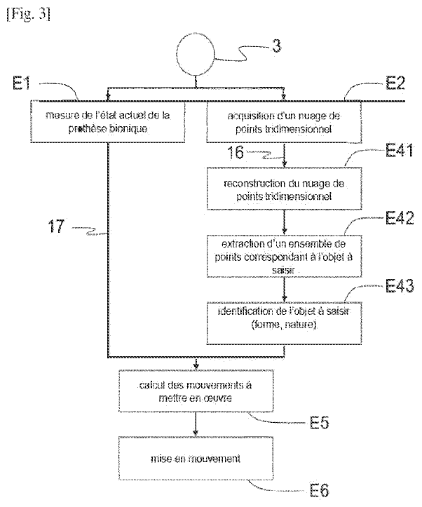

With reference to FIGS. 2 and 3, according to another aspect of the invention, the invention relates to a method for controlling the upper or lower limb bionic prosthesis, as described previously, the method comprising, following an instruction 3 corresponding to a motion intention:
measuring E1 the current state 17 of the bionic prosthesis comprising especially measuring directional vectors of the prosthetic distal segment, the prosthetic middle segment, and, where applicable, the prosthetic proximal segment, in particular by the set of accelerometers;
acquiring E2 a three-dimensional point cloud 16 of the environment of the bionic prosthesis by said at least one depth camera;
processing the three-dimensional point cloud 16 by the control unit;
calculating E5 the motions to be implemented by said at least one motor-driven hinge device by the control unit;
moving E6 the bionic prosthesis.

Furthermore, a possible acquisition frequency of said at least one depth camera is 5 Hz. The choice of the acquisition frequency results especially from a compromise between obtaining a sufficient number of data to have a sufficient representation of the environment, while limiting the number of data to be processed in order to reduce the processing time and therefore save on the battery of the control unit.

Calculating the motions to be performed by said at least one motor-driven hinge device by the control unit may in particular consist in optimizing the motions according to the different degrees of freedom of said at least one motor-driven hinge device.

The bionic prosthesis forming a bionic lower limb prosthesis, according to a first embodiment of the invention, is described in more detail below.

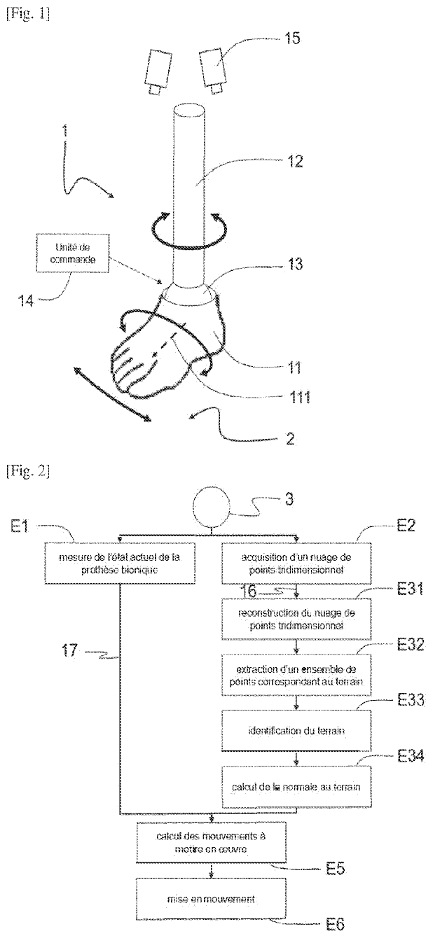

With reference to [FIG. 1], the bionic lower limb prosthesis 1 according to the first embodiment of the invention comprises a prosthetic foot 11 at a free end of the bionic prosthesis 1 and adapted to contact a ground 2; a prosthetic tibia 12; at least one motor-driven hinge device 13, a first of said at least one motor-driven hinge device 13 being mechanically connected to the prosthetic foot 11 on the one hand and to the prosthetic tibia 12 on the other hand; a control unit 14 configured to control said at least one motor-driven hinge device 13 from control signals corresponding to a motion intention; at least one depth camera 15 adapted to generate a three-dimensional point cloud of the environment of the prosthetic foot 11, comprising the ground 2, the control unit 14 being configured to process the three-dimensional point cloud in order to detect the ground 2 and to control said at least one motor-driven hinge device 13 so as to adapt the positioning of the prosthetic foot 11 relative to the ground 2.

In particular, the ground is defined as a surface on which the amputee takes support to move. The surface may especially be planar such as a floor or step, or inclined. In addition, the surface may also be uneven and comprise obstacles, or roughnesses (holes, stones, etc.) that it is advantageous to identify.

In addition, said at least one depth camera may be adapted to view the other leg of the amputee in addition to the ground.

Besides, said at least one depth camera 15 can consist of two depth cameras configured so as to cover respectively a front field and a rear field of the environment of the prosthetic foot 11 as represented in [FIG. 1]. Such a camera configuration makes it possible to perceive the environment when walking forwards and backwards. Thus, the two depth cameras can be positioned, for example, respectively at the front and rear of the bionic prosthesis, or on either side of the bionic lower limb prosthesis.

In addition, said at least one depth camera preferably has a fixed origin relative to the bionic lower limb prosthesis.

In a first example, the bionic lower limb prosthesis according to the invention is adapted to a case of tibial amputation, that is, below the knee. Then, only the first motor-driven hinge device, corresponding to the ankle, is controlled by the control unit. Then, the first motor-driven hinge device may advantageously have three degrees of freedom represented by bidirectional arrows in [FIG. 1]. The control unit therefore controls a total of three degrees of freedom.

In a second example, the bionic lower limb prosthesis according to the invention is adapted to a case of transfemoral amputation, that is, above the knee. The bionic lower limb prosthesis then advantageously comprises a prosthetic femur. In addition, for a bionic lower limb prosthesis, said at least one motor-driven hinge device comprises in particular the second motor-driven hinge device being mechanically connected to the prosthetic tibia on the one hand and to the prosthetic femur on the other hand. In other words, the second motor-driven hinge device corresponds to the knee joint.

In this second example, the degrees of freedom of the second motor-driven hinge device are added to the three degrees of freedom of the first motor-driven hinge device. The present invention advantageously makes it possible to more precisely adapt the height at which the bionic lower limb prosthesis, and especially the second motor-driven hinge device corresponding to the knee, have to be lifted to avoid clinging on the ground, in particular for non-flat ground.

Furthermore, the prosthetic foot 11 especially has a directional vector of the foot 111 oriented substantially in a longitudinal direction of the prosthetic foot 11 as illustrated in [FIG. 1]. The bionic lower limb prosthesis 1 may then comprise a set of accelerometers capable of measuring the directional vector of the foot 111, the set of accelerometers preferably comprising two accelerometers. The directional vector of the foot 111 can especially be defined relative to a reference system defined by three axes corresponding to a reference position of the prosthetic foot 11.

With reference to [FIG. 2], the method for controlling the bionic lower limb prosthesis, following an instruction 3 corresponding to a motion intention, comprises:
measuring E1 the current state 17 of the bionic lower limb prosthesis comprising measuring the directional vector of the foot especially by the set of accelerometers;
acquiring E2 the three-dimensional point cloud 16 by said at least one depth camera;
processing the three-dimensional point cloud 16 by the control unit comprising extracting E32 a set of points from the three-dimensional point cloud 16 corresponding to the ground and calculating the normal E34 to a surface defined by said set of points, referred to as the normal to the ground;
calculating E5 the motions to be implemented by said at least one motor-driven hinge device by the control unit 14 so that the directional vector of the foot is substantially perpendicular to the normal to the ground;
moving E6 the bionic prosthesis.

In addition, said at least one depth camera is, preferably, activated only when walking and otherwise is at rest.

Especially, extracting a set of points from the three-dimensional point cloud corresponding to the ground can consist in running a planar surface detection algorithm and then selecting the planar surface with the largest number of points. Thus, the invention makes it possible to advantageously detect planar and inclined surfaces.

Besides, the method for controlling the bionic lower limb prosthesis may comprise identifying the nature of the ground E33, as illustrated in [FIG. 2], for example a stair step. Identifying the nature of the ground can especially be carried out by means of learning algorithms. Within the context of a step on stairs, the first motor-driven hinge device, corresponding to the ankle, has to incline forward to avoid falling while still being flat in order to ensure stable support. Thus, the forward inclination is a restriction to be taken into account when calculating the motions to be performed by said at least one motor-driven hinge device by the control unit.

In addition, as illustrated in [FIG. 2], the method according to the invention may comprise a step of reconstructing E31 the three-dimensional point cloud 16 comprising assembling a plurality of three-dimensional point clouds 16 corresponding to successively acquiring three-dimensional point clouds by said at least one depth camera, the reconstruction step being especially performed prior to calculating the normal to the ground E34. Thus, the reconstruction step E31 can be performed before or after the extraction of E32 and the identification of the ground E33. The reconstruction step advantageously makes it possible to reconstitute the zones obstructed by the prosthetic foot in order to obtain an improved perception of the ground. Thus, any uncertainties related to the calculation of the normal to the ground are reduced.

The reconstruction step may preferably comprise running a SLAM (“Simultaneous Localization And Mapping”) algorithm.

Calculating the motions to be performed by said at least one motor-driven hinge device by the control unit may in particular consist in optimizing the motions according to the different degrees of freedom of said at least one motor-driven hinge device. The objective is to ensure a positioning of the prosthetic foot substantially parallel to the ground for improved grip while minimizing the motions required so as to reduce fatigue and consumption of the motors of said at least one motor-driven hinge device.

The bionic prosthesis forming a bionic upper limb prosthesis, according to a second embodiment of the invention, is described in more detail below.

The bionic upper limb prosthesis according to the second embodiment of the invention comprises a prosthetic hand at a free end of the bionic prosthesis and adapted to contact an object to be gripped; a prosthetic forearm; at least one motor-driven hinge device, a first of said at least one motor-driven hinge device being mechanically connected to the prosthetic hand on the one hand and to the prosthetic forearm on the other hand; a control unit configured to control said at least one motor-driven hinge device from control signals corresponding to a motion intention; at least one depth camera adapted to generate a three-dimensional point cloud of the environment of the prosthetic hand, comprising the object to be gripped, the control unit being configured to process the three-dimensional point cloud in order to detect the object to be gripped and to control said at least one motor-driven hinge device so as to adapt the positioning of the prosthetic hand relative to the object to be gripped.

Besides, said at least one depth camera may, preferably, be integrated between the elements corresponding to the thumb and index finger of the prosthetic hand, into the palm of the prosthetic hand, or on the face opposite to the palm of the prosthetic hand. In addition, it is also contemplatable to use multiple depth cameras, each covering a field of the environment.

In a third example, the bionic upper limb prosthesis according to the invention is adapted to a case of forearm amputation, that is, below the elbow. The first motor-driven hinge device, corresponding to the wrist joint, is controlled by the control unit. Then, the first motor-driven hinge device may advantageously have three degrees of freedom. In addition, further additional hinge devices, especially corresponding to finger joints, may be added to the first motor-driven hinge device.

In a fourth example, the bionic upper limb prosthesis according to the invention is adapted to a case of arm amputation, that is, above the elbow. The bionic upper limb prosthesis then advantageously comprises a prosthetic arm. In addition, for a bionic upper limb prosthesis, said at least one motor-driven hinge device comprises in particular the second motor-driven hinge device being mechanically connected to the prosthetic forearm on the one hand and to the prosthetic arm on the other hand. In other words, the second motor-driven hinge device corresponds to the elbow joint. In this second example, the degrees of freedom of the second motor-driven hinge device are added to the three degrees of freedom of the first motor-driven hinge device.

The present invention advantageously makes it possible to more precisely adapt the position of the prosthetic hand to the object to be gripped, thus enabling improved accuracy when gripping an object and making the gripping more natural for the user of the prosthesis.

With reference to [FIG. 3], the method for controlling the bionic upper limb prosthesis, following an instruction 3 corresponding to an intention to move, comprises:
measuring E1 the current state 17 of the bionic upper limb prosthesis comprising measuring a directional vector of the hand, especially by the set of accelerometers;
acquiring E2 the three-dimensional point cloud 16 by said at least one depth camera;
processing the three-dimensional point cloud 16 by the control unit comprising extracting E42 a set of points from the three-dimensional point cloud 16 corresponding to the object to be gripped and identifying the object to be gripped E43, especially the nature and shape of the object to be gripped;
calculating E5 the motions to be implemented by said at least one motor-driven hinge device by the control unit so as to ensure the gripping of the object by the prosthetic hand;
moving E6 the bionic prosthesis.

Especially, extracting a set of points from the three-dimensional point cloud corresponding to the object to be gripped and identifying the object to be gripped can be carried out by means of learning algorithms.

In addition, as illustrated in [FIG. 3], the method according to the invention may comprise a step of reconstructing E41 the three-dimensional point cloud 16 comprising assembling a plurality of three-dimensional point clouds 16 corresponding to successively acquiring three-dimensional point clouds by said at least one depth camera. The reconstruction step may preferably comprise running a SLAM (“Simultaneous Localization And Mapping”) algorithm. The reconstruction step advantageously makes it possible to reconstitute the obstructed zones in order to obtain an improved perception of the object to be gripped, and possibly to limit the number of depth cameras used.

In summary, the present invention provides a bionic prosthesis capable of perceiving the environment by the use of depth cameras and a method for controlling said bionic prosthesis. Thus, the invention has the substantial gain of reducing the mental and physical cost of using a bionic prosthesis. For example, the lower limb amputee no longer has to worry about whether the bionic prosthesis is raised enough to avoid tripping on stairs or non-flat ground. Similarly, the upper limb amputee no longer has to worry about the position and the force exerted by the prosthetic hand when gripping an object. Consequently, the present invention allows a more natural, intuitive, and stable movement. In addition, the use of the prosthesis is simplified.

In addition, the present invention enables improved control of the different degrees of freedom of motor-driven hinge devices, and enables control of bionic prostheses having a greater number of degrees of freedom. By way of example, according to the state of the art, conventional bionic lower limb prostheses generally comprise only one degree of freedom at the first motor-driven hinge device corresponding to the ankle joint. By means of the invention, it is possible to use a first motor-driven hinge device having, for example, three degrees of freedom. Similarly, conventional bionic upper limb prostheses generally comprise only one degree of freedom at the first motor-driven hinge device corresponding to the wrist hinge in the form of an infinite rotation. The number of degrees of freedom can also be increased in the latter case. Thus, the invention provides a wider range of motion, allowing, within the context of a bionic lower limb prosthesis, balance to be maintained when climbing and descending stairs or even when walking backwards, and within the context of a bionic upper limb prosthesis, to be gripped delicate objects such as a fruit or a stemmed glass.

1-8. (canceled)

9. A bionic prosthesis usable on a lower limb or an upper limb, the bionic prosthesis comprising:
a prosthetic distal segment of the lower or upper limb, in other words, a prosthetic foot or, respectively, a prosthetic hand, at a free end of the bionic prosthesis, adapted to contact a ground or, respectively, an object to be gripped;
a prosthetic middle segment, in other words, a prosthetic tibia or, respectively, a prosthetic forearm;
at least one motor-driven hinge device, a first of said at least one motor-driven hinge device being mechanically connected to the prosthetic distal segment on the one hand and to the prosthetic middle segment on the other hand;
a control unit configured to control said at least one motor-driven hinge device from control signals corresponding to a motion intention;
wherein the bionic prosthesis comprises at least one depth camera adapted to generate a three-dimensional point cloud of the environment of the bionic prosthesis, comprising the ground or, respectively, the object to be gripped, the control unit being configured to process the three-dimensional point cloud in order to detect the ground or, respectively, the object to be gripped, and to control said at least one motor-driven hinge device so as to adapt the positioning of the prosthetic distal segment relative to the ground or, respectively, relative to the object to be gripped, the control unit being configured to perform a reconstruction of the three-dimensional point cloud comprising assembling a plurality of three-dimensional point clouds corresponding to successively acquiring three-dimensional point clouds by said at least one depth camera.

10. The bionic prosthesis according to claim 9, forming a bionic lower limb prosthesis, comprising:
a prosthetic foot at a free end of the bionic prosthesis, adapted to contact a ground;
a prosthetic tibia;
at least one motor-driven hinge device, a first of said at least one motor-driven hinge device being mechanically connected to the prosthetic foot on the one hand and to the prosthetic tibia on the other hand;
a control unit configured to control said at least one motor-driven hinge device from control signals corresponding to a motion intention;
wherein the bionic prosthesis comprises at least one depth camera adapted to generate a three-dimensional point cloud of the environment of the prosthetic foot, comprising the ground, the control unit being configured to process the three-dimensional point cloud in order to detect the ground and to control said at least one motor-driven hinge device so as to adapt the positioning of the prosthetic foot relative to the ground.

11. The bionic prosthesis according to the preceding claim, wherein said at least one depth camera consists of two depth cameras configured so as to cover respectively a front field and a rear field of the environment of the prosthetic foot.

12. The bionic prosthesis according to claim 9, wherein it comprises a myoelectric sensor system configured to generate the control signals corresponding to a motion intention.

13. The bionic prosthesis according to claim 10, the prosthetic foot having a directional vector of the foot oriented substantially in a longitudinal direction of the prosthetic foot, wherein it comprises a set of accelerometers capable of measuring the directional vector of the foot.

14. The bionic prosthesis according to claim 10, wherein it comprises a prosthetic femur, said at least one motor-driven hinge device comprising a second motor-driven hinge device being mechanically connected to the prosthetic tibia on the one hand and to the prosthetic femur on the other hand.

15. A method for controlling a bionic lower or upper limb prosthesis comprising a prosthetic distal segment of the lower or upper limb, in other words a prosthetic foot or, respectively, a prosthetic hand, configured to contact a ground or, respectively, an object to be gripped; a prosthetic middle segment, in other words a prosthetic tibia or, respectively, a prosthetic forearm; where applicable, a prosthetic proximal segment, in other words, a prosthetic femur or, respectively, a prosthetic arm; at least one motor-driven hinge device; a control unit configured to control the motor-driven hinge device; at least one depth camera adapted to generate a three-dimensional point cloud of the environment of the bionic prosthesis comprising the ground or, respectively, the object to be gripped, the method, following an instruction corresponding to a motion intention, comprising:
measuring the current state of the bionic prosthesis, comprising measuring directional vectors of the prosthetic distal segment, the prosthetic middle segment, and, where applicable, the prosthetic proximal segment by a set of accelerometers;
acquiring a three-dimensional point cloud of the environment of the bionic prosthesis by said at least one depth camera;
a step of reconstructing the three-dimensional point cloud comprising assembling a plurality of three-dimensional point clouds corresponding to successively acquiring three-dimensional point clouds by said at least one depth camera;
processing the three-dimensional point cloud by the control unit;
calculating the motions to be implemented by said at least one motor-driven hinge device by the control unit;
moving the bionic prosthesis.

16. The method for controlling the bionic prosthesis according to claim 15, adapted for a bionic lower limb prosthesis comprising a prosthetic foot configured to contact a ground, a prosthetic tibia, at least one motor-driven hinge device, a control unit configured to control the motor-driven hinge device, at least one depth camera adapted to generate a three-dimensional point cloud of the environment of the bionic prosthesis comprising the ground, the method, following an instruction corresponding to a motion intention, comprising:
measuring the current state of the bionic prosthesis, comprising measuring a directional vector of the foot oriented substantially in a longitudinal direction of the prosthetic foot by a set of accelerometers;
acquiring the three-dimensional point cloud of the environment of the bionic prosthesis by said at least one depth camera;
processing the three-dimensional point cloud by the control unit comprising extracting a set of points from the three-dimensional point cloud corresponding to the ground, the step of reconstructing the three-dimensional point cloud, and calculating the normal to a surface defined by said set of points, referred to as the normal to the ground;
calculating the motions to be implemented by said at least one motor-driven hinge device by the control unit so that the directional vector of the foot is substantially perpendicular to the normal to the ground;
moving the bionic prosthesis.
